(19) United States
(12) Patent Application Publication
(10) Pub. No.: US 2020/0179151 A1
(43) Pub. Date: Jun. 11, 2020

(54) WEARABLE DEVICE FOR TREATING CARPAL TUNNEL SYSDROME

(71) Applicant: LUNEMED SRL, Samarate (IT)

(72) Inventors: Luca Negretto, Samarate (IT); Severino Negretto, Samarate (IT)

(21) Appl. No.: 16/620,903

(22) PCT Filed: Jun. 18, 2018
(86) PCT No.: PCT/EP2018/066052

§ 371 Date: Dec. 10, 2019

(30) Foreign Application Priority Data
Jun. 22, 2017 (IT) 102017000069456

(51) Int. Cl. A61F 5/01

(52) U.S. Cl. CPC A61F 5/0118

(57) ABSTRACT

A wearable device (10) for treating carpal tunnel syndrome, which comprises a main body (12) configured to accommodate a hand (30), the main body (12) comprising a back (54) and a palm (56); the wearable device (10) comprises an elastic band (16), a first end (15) of the elastic band (16) being coupled to a first lateral end (24) of the main body (12), fixing means (19) which are adapted to couple a second end (17) of the elastic band (16) to a second lateral end (22) of the main body (12), and an elastic pad (14) arranged on the back (54) of the main body (12); the elastic band (16) rests on the elastic pad (14) and is adapted to induce a traction between the first lateral end (24) and the second lateral end (22) of the main body (12); the elastic pad (14) is adapted to convert the traction induced by the elastic band (16) to a pressure applied to the metacarpal bones (42) of the hand (30).

The present invention relates to a wearable device for treating carpal tunnel syndrome. The wearable device described herein is particularly, although not exclusively, useful and practical in a medical/health context, for treating people affected by carpal tunnel syndrome.

Carpal tunnel syndrome (or CTS) is a neuropathy caused by the irritation or compression of the median nerve, which is the main nerve bundle of the hand, in its passage through the carpal channel (or tunnel).

This nervous disease is characterized by symptoms such as for example pain, tingling, and numbness in the user's hand and/or fingers. Sometimes the above symptoms are not confined to the hand alone, but can also affect the forearm.

In addition, carpal tunnel syndrome can give rise to manifestation of further symptoms, such as for example difficulty in bending the fingers of the user's hand, weakening of the muscles of the hand and/or reduction in sensitivity of the fingers of the hand.

In the last few decades medical/health research has made it possible to identify the links between carpal tunnel syndrome (or STC) and certain exacerbating situations. In particular, carpal tunnel syndrome tends to develop more in persons who perform repetitive movements with their hands or who use their hands for precision work.

For example, the frequent use of a mouse or keyboard of a personal computer, the use of musical instruments, such as for example the guitar or the drums, or the practicing of some sports, such as for example bowling or tennis, cause micro-injuries to the wrist and a compression of the median nerve, contributing to the onset of carpal tunnel syndrome.

Other factors like heredity, hormonal changes associated with pregnancy or underlying general disease conditions, including diabetes, can be the cause of carpal tunnel syndrome.

If diagnosis is early, carpal tunnel syndrome can be easily cured without the need for surgery. In more serious cases surgery is necessary, but it is not without risk and it does not guarantee certain and complete recovery. Such surgery, in fact, can cause complications and relapses.

For this reason, it is advisable for the user to tackle and treat carpal tunnel syndrome as soon as possible, by way of adapted therapeutic treatments.

Nowadays various therapeutic treatments are known for treating carpal tunnel syndrome, which can be selected on the basis of the seriousness of the condition and on the duration of the corresponding symptoms. For example, in addition to the surgery mentioned above, wearable devices are known such as semi-rigid wrist guards, slats, or braces.

These conventional wearable devices prevent any movement of the wrist, keeping it in a neutral position and preventing any involuntary movements of the wrist that might for example occur during sleep. By virtue of immobilizing the wrist, these conventional wearable devices prevent further inflammation of the median nerve and consequent worsening of the symptoms.

However, such conventional solutions are not devoid of drawbacks, among which is the fact that, during their application, they determine a stiffening of the wrist and a decrease in sensitivity of the fingers of the hand, causing a consequent decrease in the capacity for practical use of the hand.

Another drawback of such conventional solutions consists in that they are adapted only for nighttime use. A daytime use of such conventional solutions, in fact, entails considerable impediment to the various daily activities carried out by the user.

The aim of the present invention is to overcome the limitations of the known art described above, by devising a wearable device for treating carpal tunnel syndrome that makes it possible to alleviate the pressure on the median nerve, the inflammation of which is responsible for the pain.

Within this aim, an object of the present invention is to provide a wearable device for treating carpal tunnel syndrome that enables daytime use of such wearable device by the user, without preventing the carrying out of daily activities.

Another object of the present invention is to devise a wearable device for treating carpal tunnel syndrome that enables a gradual recovery from carpal tunnel syndrome.

Another object of the present invention is to provide a wearable device for treating carpal tunnel syndrome that enables a decompression of the inflamed median nerve, by favoring a gradual disinflammation of the median nerve proper with consequent alleviation or disappearance of the pain.

Another object of the present invention is to devise a wearable device for treating carpal tunnel syndrome that enables self-administered use, being adjustable directly by the user according to the perception of pain and of tingling.

Another object of the present invention is to provide a wearable device for treating carpal tunnel syndrome that is not disabling, thus allowing the user to carry out normal daily activities.

Another object of the present invention is to devise a wearable device for treating carpal tunnel syndrome that allows non-invasive use, it being capable of being worn or removed easily by the user according to needs.

Another object of the present invention is to provide a wearable device for treating carpal tunnel syndrome that is highly reliable, easily and practically implemented, and low cost when compared to the known art.

This aim and these and other objects which will become better apparent hereinafter are achieved by a wearable device for treating carpal tunnel syndrome, which comprises a main body configured to accommodate a hand, said main body comprising a back and a palm, characterized in that said wearable device comprises an elastic band, a first end of said elastic band being coupled to a first lateral end of said main body, means for fixing which are adapted to couple a second end of said elastic band to a second lateral end of said main body, and an elastic pad arranged on said back of said main body; said elastic band rests on said elastic pad and is adapted to induce a traction between said first lateral end and said second lateral end of said main body; said elastic pad is adapted to convert said traction induced by said elastic band to a pressure applied to the metacarpal bones of said hand.

Further characteristics and advantages of the invention will become better apparent from the detailed description of a preferred, but not exclusive, embodiment of the wearable device for treating carpal tunnel syndrome according to the invention, illustrated by way of non-limiting example in the accompanying drawings wherein:

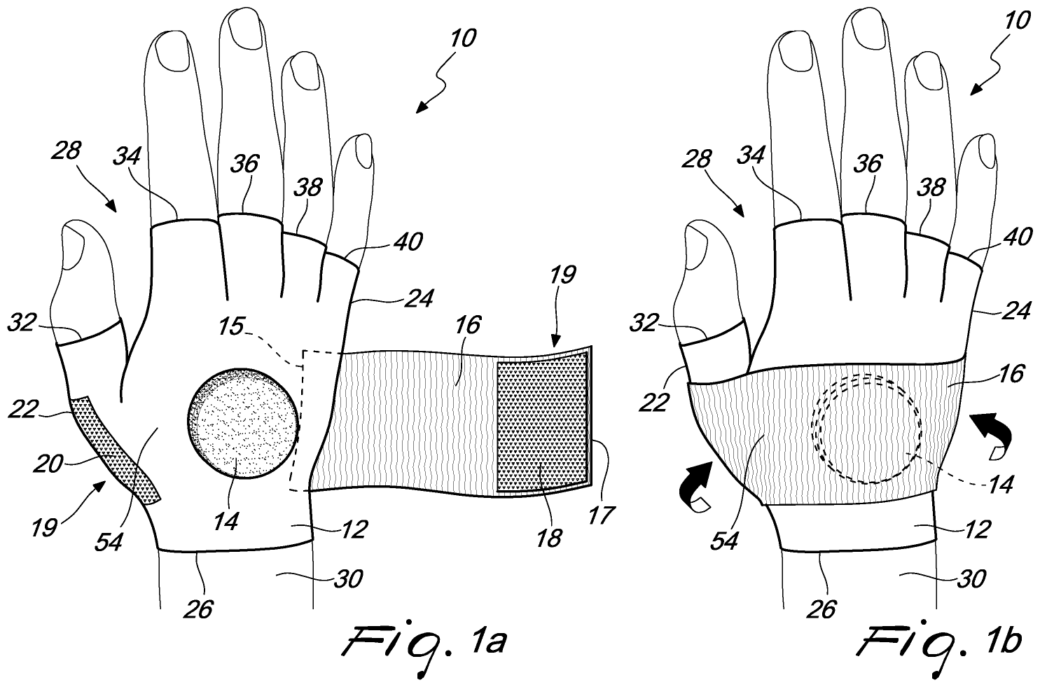

FIGS. 1a and 1b show the rear or dorsal part of an embodiment of the wearable device for treating carpal tunnel syndrome, according to the invention, respectively in the open and closed configurations;

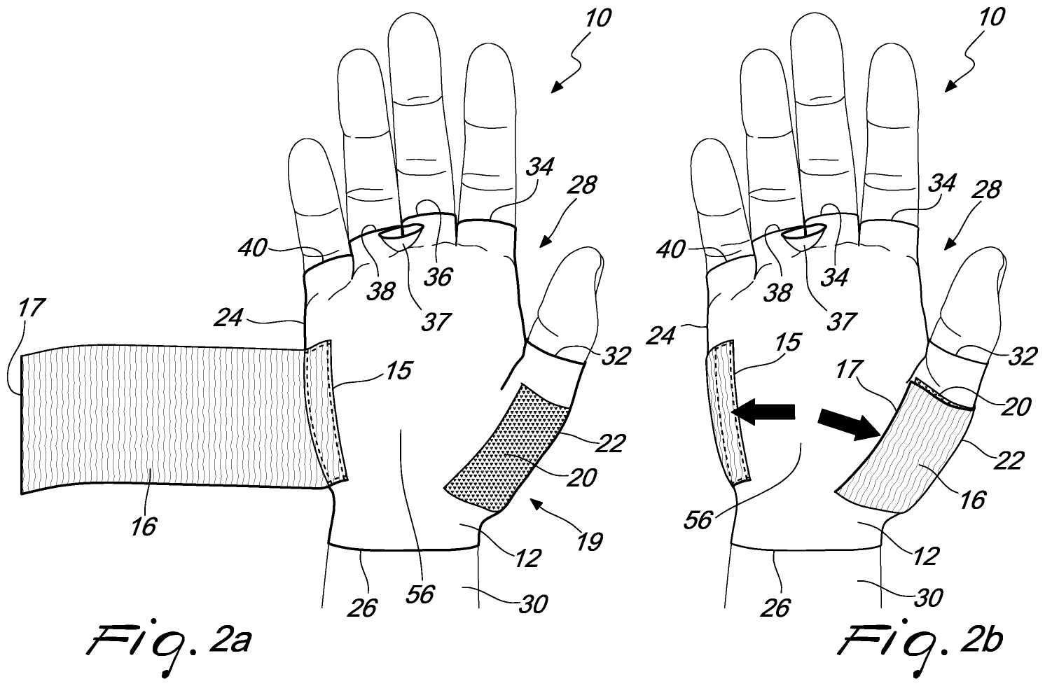

FIGS. 2a and 2b show the front or palm part of an embodiment of the wearable device for treating carpal tunnel syndrome, according to the invention, respectively in the open and closed configurations;

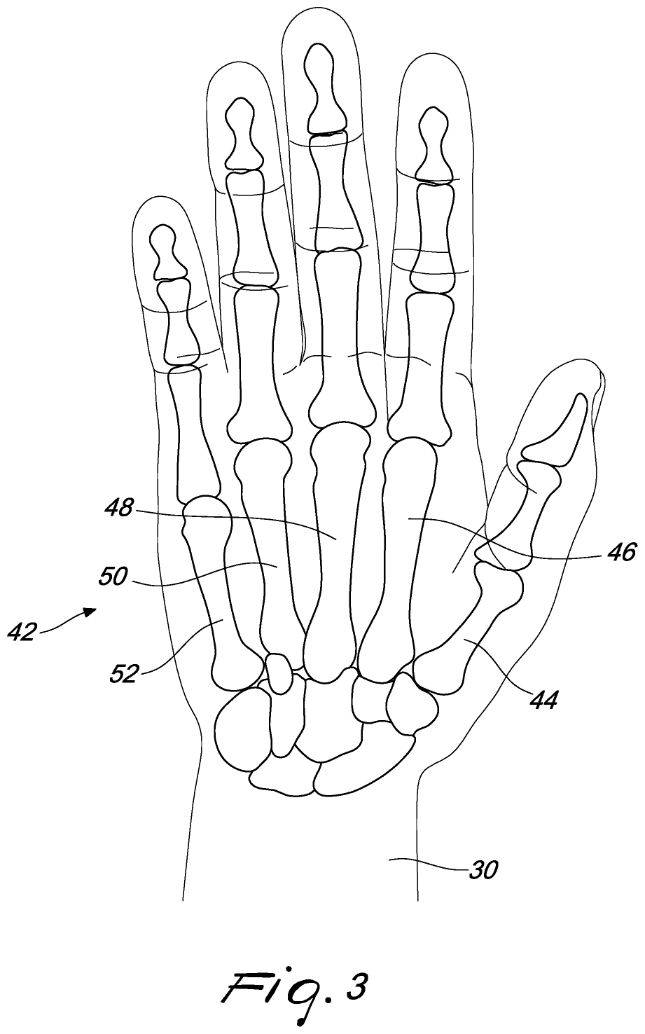

FIG. 3 shows the skeletal structure of a user's hand.

With reference to the figures, the wearable device for treating carpal tunnel syndrome according to the invention, generally designated by the reference numeral 10, substantially comprises a main body 12, an elastic band 16 associated with the main body 12, fixing means 19, and an elastic pad 14 which is associated with the main body 12.

The main body 12 of the wearable device 10 according to the invention is hollow and is configured to receive a hand 30, i.e. it is contoured to be worn on a hand 30. The main body 12 is not rigid, but deformable, thus facilitating the mobility of the user's hand 30. Preferably, the main body 12 completely envelops the metacarpal bones 42 of the hand 30. Preferably, the main body 12 is a glove. This does not rule out the possibility that the main body 12 can also be provided in a form other than a glove.

The main body 12 comprises a back 54 or rear part of the main body 12, and a palm 56 or front part of the main body 12. In an embodiment of the invention, the main body 12 further comprises an extractor 37, in the form of a tab or a flap, for example made of fabric, which is adapted to facilitate the extraction of the wearable device 10, in particular of the main body 12 proper, from the hand 30.

Preferably, the back 54 is made of spandex, or Lycra. Alternatively, the back 54 can be made of elastic jersey. Advantageously, the back 54 can be made of an elasticized double fabric, so that the elastic pad 14 can be arranged and coupled between the two layers of the fabric.

Preferably, the palm 54 is made of a fabric with an insert made of non-slip material, so as to facilitate and increase the gripping of objects, such as for example a non-woven fabric made of polyester (e.g. polyethylene terephthalate) with a non-slip insert made of polyvinyl chloride (PVC). Advantageously, the palm 54 can be made of a friction-resistant fabric, such as for example Alcantara.

The main body 12 comprises a lower opening 26. The lower opening 26 is adapted for the passage of the hand 30. The lower opening 26 is preferably circular in cross-section. The lower opening 26 allows the user to insert the hand 30 affected by carpal tunnel syndrome into the main body 12.

The main body 12 comprises an upper end 28. The upper end 28 comprises at least one upper opening which is adapted for the passage of the fingers of the hand 30. The upper end 28 comprises an upper opening 32 which is adapted for the passage of the thumb of the hand 30. The upper end 28 comprises five upper openings 32, 34, 36, 38, 40. The upper openings 32, 34, 36, 38, 40 are preferably circular in cross-section. The upper openings 32, 34, 36, 38, 40 allow the passage of the individual fingers of the hand 30. The upper openings 32, 34, 36, 38, 40 facilitate the mobility of the fingers of the hand 30. Such upper openings 32, 34, 36, 38, 40 correspond respectively to the thumb, the index finger, the middle finger, the ring finger and the little finger of the hand 30.

The upper opening 32 is arranged proximate to the first fixing means 20 of the main body 12, while the upper opening 40 is arranged proximate to the point of association of the elastic band 16 with the main body 12.

In a different embodiment of the wearable device 10 for treating carpal tunnel syndrome according to the invention, the upper opening 32 is arranged proximate to the point of association of the elastic band 16 with the main body 12, while the upper opening 40 is arranged proximate to the first fixing means 20 of the main body 12.

The main body 12 comprises a first lateral end 24 and a second lateral end 22. The first lateral end 24 of the main body 12 is coupled to a first end 15 of the elastic band 16, for example by way of high-strength stitching. The second lateral end 22 of the main body 12 comprises first fixing means 20.

In an embodiment of the wearable device 10 for treating carpal tunnel syndrome according to the invention, the first lateral end 24 of the main body 12 is arranged proximate to the fifth metacarpal bone 52 of the hand 30.

In a different embodiment of the wearable device 10 for treating carpal tunnel syndrome according to the invention, the first lateral end 24 of the main body 12 is arranged proximate to the first metacarpal bone 44 of the hand 30.

In an embodiment of the wearable device 10 for treating carpal tunnel syndrome according to the invention, the second lateral end 22 of the main body 12 is arranged proximate to the first metacarpal bone 44 of the hand 30.

In a different embodiment of the wearable device 10 for treating carpal tunnel syndrome according to the invention, the second lateral end 22 of the main body 12 is arranged proximate to the fifth metacarpal bone 52 of the hand 30.

The elastic band 16 of the wearable device 10 according to the invention preferably extends longitudinally. For example, the elastic band 16 can be made of elasticized fabric. The elastic band 16 rests at least partially on the elastic pad 14. The elastic band 16 is adapted to induce a traction between the first lateral end 24 of the main body 12 and the second lateral end 22 of the main body 12. As a consequence, the elastic band 16 is adapted to induce a traction between the first metacarpal bone 44 of the hand 30 and the fifth metacarpal bone 52 of the hand 30.

A first end 15 of the elastic band 16 is coupled to the first lateral end 24 of the main body 12, for example by way of high-strength stitching. A second end 17 of the elastic band 16, which is opposite from the first end 15, comprises second fixing means 18.

With reference to FIGS. 1a and 2a (the open configuration of the wearable device 10), the elastic band 16 of the wearable device 10 according to the invention is coupled to the first lateral end 24 of the main body 12. The first end 15 of the elastic band 16, which is coupled to the first lateral end 24 of the main body 12, is opposite from the second end 17 of the elastic band 16 which comprises the second fixing means 18.

With reference to FIGS. 1b and 2b (the closed configuration of the wearable device 10), the elastic band 16 of the wearable device 10 according to the invention is coupled to the first lateral end 24 of the main body 12 and, by way of the fixing means 19, to the second lateral end 22 of the main body 12.

The fixing means 19 of the wearable device 10 according to the invention are adapted to establish a temporary coupling between the second end 17 of the elastic band 16 and the second lateral end 22 of the main body 12. Advantageously, the position of the coupling established by the fixing means 19 is adjustable by the user, so as to consequently adjust both the tension of the elastic band 16 and the traction exerted by the elastic band 16. The fact that the user can adjust the tension of the elastic band 16 is an important aspect, because in practice different tensions are necessary according to the state of inflammation of the median nerve and to the pain perceived by the user during use of the wearable device 10.

Preferably, the fixing means 19 of the wearable device 10 according to the invention comprise hook-and-loop fixing elements, or Velcro. The fixing means 19 comprise first fixing means 20 of the main body 12, and second fixing means 18 of the elastic band 16.

With reference to FIGS. 1b and 2b (the closed configuration of the wearable device 10), the first fixing means 20 of the main body 12 are coupled to the second fixing means 18 of the elastic band 16. The first fixing means 20 are arranged at the second lateral end 22 of the main body 12. The first fixing means 20 are arranged proximate to the first metacarpal bone 44 of the hand 30.

With reference to FIGS. 1b and 2b (the closed configuration of the wearable device 10), the second fixing means 18 of the elastic band 16 are coupled to the first fixing means 20 of the main body 12. The second fixing means 18 are arranged at the second end 17 of the elastic band 16.

In an embodiment of the wearable device 10 according to the invention, the first fixing means 20 are arranged between the back 54 of the main body 12 and the palm 56 of the main body 12, in order to facilitate the user in the adjustment of the position of the coupling established by the fixing means 19, and consequently in the adjustment both of the tension of the elastic band 16 and of the traction exerted by the elastic band 16.

The elastic pad 14 of the wearable device 10 according to the invention is arranged and coupled on the back 54 of the main body 12. The elastic pad 14 is arranged proximate to the lower opening 26 of the main body 12. The elastic pad 14 is made of elastic material, for example neoprene. The elastic pad 14, and therefore also the elastic material with which it is provided, has a density comprised between 160 and 200 kg/m³, preferably comprised between 170 and 190 kg/m³, and even more preferably equal to 180 kg/m³.

The height of the elastic pad 14 is comprised between 8 and 12 millimeters, and preferably is equal to 10 millimeters. In a preferred embodiment of the invention, the shape of the elastic pad 14 is circular, with a diameter of length comprised between 40 and 50 millimeters. The circular shape, with no corners, allows the elastic band 16 to rest on the elastic pad 14, covering it completely.

In another embodiment of the invention, the shape of the elastic pad 14 is square, with a side of length comprised between 40 and 50 millimeters. In an additional embodiment of the invention, the shape of the elastic pad 14 is rectangular.

The elastic pad 14 undergoes the traction induced by the elastic band 16 between the first lateral end 24 and the second lateral end 22 of the main body 12, i.e. between the first metacarpal bone 44 and the fifth metacarpal bone 52 of the hand 30. This traction induced by the elastic band 16 is comprised between 0.6 and 1.2 kg. In practice, the elastic pad 14 acts as a fulcrum for the elastic band 16.

The elastic pad 14, in undergoing this traction of the elastic band 16, exerts a consequent pressure on the metacarpal bones 42 of the hand 30 of a user, in particular on the second, third and fourth metacarpal bone 46, 48 and 50. The elastic pad 14 is adapted to convert the traction induced by the elastic band 16 to a pressure exerted on the metacarpal bones 42 of the hand 30 of a user, in particular on the second, third and fourth metacarpal bone 46, 48 and 50.

Advantageously, the elastic pad 14 is arranged at the second, third and fourth metacarpal bone 46, 48 and 50 of the metacarpal bones 42 of the hand 30 of the user.

The elastic pad 14 is arranged at the elastic band 16 of the wearable device 10. With reference to FIG. 1a (the open configuration of the wearable device 10), the elastic pad 14 is particularly distinguishable by the user. With reference to FIG. 1b (the closed configuration of the wearable device 10), the elastic band 16 is superimposed on the elastic pad 14, and therefore the elastic pad is arranged completely underneath the elastic band 16.

The combined effect of the traction induced by the elastic band 16 and of the pressure exerted by the elastic pad 14 produces a slight arching, particularly upward (when looking at the back of the hand 30), of the metacarpal bones 42 of the hand 30 of the user. This arching in turn generates a slight stretching of the transverse carpal ligament, a dilation of the carpal tunnel and a consequent decompression of the median nerve of the hand 30.

Operation of an embodiment of the wearable device 10 for treating carpal tunnel syndrome according to the invention is described below.

The user inserts the hand 30 into the lower opening 26 of the main body 12 of the wearable device 10. The user inserts the fingers of the hand 30 into the upper openings 32, 34, 36, 38, 40. The user grips the elastic band 16, in particular its second end 17, and couples the second fixing means 18 of the elastic band 16 to the first fixing means 20 of the main body 12. The elastic band 16 rests at least partially on the elastic pad 14.

The coupling between the second fixing means 18 of the elastic band 16, and the first fixing means 20 of the main body 12 enables the elastic band 16 to induce a traction between the first lateral end 24 and the second lateral end 22 of the main body 12, i.e. between the first metacarpal bone 44 and the fifth metacarpal bone 52 of the hand 30.

The elastic pad 14, in undergoing this traction of the elastic band 16, exerts a consequent pressure on the metacarpal bones 42 of the hand 30 of a user, in particular on the second, third and fourth metacarpal bone 46, 48 and 50. The elastic pad 14 is adapted to convert the traction induced by the elastic band 16 to a pressure exerted on the metacarpal bones 42 of the hand 30 of a user, in particular on the second, third and fourth metacarpal bone 46, 48 and 50.

The adjustment of the traction induced by the elastic band 16 is performed directly by the user, by way of the fixing means 19, on the basis of the state of inflammation of the median nerve and of the pain perceived by the user during use of the wearable device 10.

In practice it has been found that the invention fully achieves the set aim and objects. In particular, it has been seen that the wearable device for treating carpal tunnel syndrome according to the invention thus conceived makes it possible to overcome the qualitative limitations of the known art, since it makes it possible to alleviate the pressure on the median nerve, the inflammation of which is responsible for the pain.

Another advantage of the wearable device for treating carpal tunnel syndrome according to the invention consists in that it enables daytime use of such wearable device by the user, without preventing the carrying out of daily activities.

Another advantage of the wearable device for treating carpal tunnel syndrome according to the invention consists in that it enables a gradual recovery from carpal tunnel syndrome.

Another advantage of the wearable device for treating carpal tunnel syndrome according to the invention consists in that it enables a decompression of the inflamed median nerve, by favoring a gradual disinflammation of the median nerve proper with consequent alleviation or disappearance of the pain.

Another advantage of the wearable device for treating carpal tunnel syndrome according to the invention consists in that it enables self-administered use, the tension of the elastic band being adjustable directly by the user according to the perception of pain and of tingling.

Another advantage of the wearable device for treating carpal tunnel syndrome according to the invention consists in that it is not disabling, thus allowing the user to carry out normal daily activities.

Another advantage of the wearable device for treating carpal tunnel syndrome according to the invention consists in that it allows non-invasive use, it being capable of being worn or removed easily by the user according to needs.

Although the wearable device for treating carpal tunnel syndrome according to the invention has been conceived in particular for treating carpal tunnel syndrome, it can also be used, more generally, for treating pains and sensitivity disorders and neuropathies that affect the hand of a user.

The invention, thus conceived, is susceptible of numerous modifications and variations, all of which are within the scope of the appended claims. Moreover, all the details may be substituted by other, technically equivalent elements.

In practice the materials employed, provided they are compatible with the specific use, and the contingent dimensions and shapes, may be any according to requirements and to the state of the art.

In conclusion, the scope of protection of the claims shall not be limited by the explanations or by the preferred embodiments illustrated in the description by way of examples, but rather the claims shall comprise all the patentable characteristics of novelty that reside in the present invention, including all the characteristics that would be considered as equivalent by the person skilled in the art.

The disclosures in Italian Patent Application No. 102017000069456 from which this application claims priority are incorporated herein by reference.

Where technical features mentioned in any claim are followed by reference signs, those reference signs have been included for the sole purpose of increasing the intelligibility of the claims and accordingly, such reference signs do not have any limiting effect on the interpretation of each element identified by way of example by such reference signs.

1. A wearable device (10) for treating carpal tunnel syndrome, which comprises a main body (12) configured to accommodate a hand (30), said main body (12) comprising a back (54) and a palm (56), characterized in that said wearable device (10) comprises an elastic band (16), a first end (15) of said elastic band (16) being coupled to a first lateral end (24) of said main body (12), fixing means (19) which are adapted to couple a second end (17) of said elastic band (16) to a second lateral end (22) of said main body (12), and an elastic pad (14) arranged on said back (54) of said main body (12); said elastic band (16) rests on said elastic pad (14) and is adapted to induce a traction between said first lateral end (24) and said second lateral end (22) of said main body (12); said elastic pad (14) is adapted to convert said traction induced by said elastic band (16) to a pressure applied to the metacarpal bones (42) of said hand (30).

2. The wearable device (10) for treating carpal tunnel syndrome according to claim 1, characterized in that said elastic pad (14) is arranged at the second, third and fourth metacarpal bone (46, 48, 50) of said metacarpal bones (42) of said hand (30), said pressure being exerted on said second, third and fourth metacarpal bone (46, 48, 50) of said metacarpal bones (42) of said hand (30).

3. The wearable device (10) for treating carpal tunnel syndrome according to claim 1, characterized in that said fixing means (19) comprise first fixing means (20) of said main body (12), and second fixing means (18) of said elastic band (16).

4. The wearable device (10) for treating carpal tunnel syndrome according to claim 3, characterized in that said first fixing means (20) of said main body (12) are arranged at said second lateral end (22) of said main body (12).

5. The wearable device (10) for treating carpal tunnel syndrome according to claim 3, characterized in that said second fixing means (18) of said elastic band (16) are arranged at said second end (17) of said elastic band (16).

6. The wearable device (10) for treating carpal tunnel syndrome according to claim 1, characterized in that said fixing means (19) comprise hook-and-loop fixing elements.

7. The wearable device (10) for treating carpal tunnel syndrome according to claim 1, characterized in that said traction induced by said elastic band (16) is comprised between 0.6 and 1.2 kg.

8. The wearable device (10) for treating carpal tunnel syndrome according to claim 1, characterized in that said elastic pad (14) is arranged at said elastic band (16).

9. The wearable device (10) for treating carpal tunnel syndrome according to claim 1, characterized in that said elastic pad (14) has a circular shape.

10. The wearable device (10) for treating carpal tunnel syndrome according to claim 9, characterized in that said elastic pad (14) has a diameter of length comprised between 40 and 50 millimeters.

11. The wearable device (10) for treating carpal tunnel syndrome according to claim 1, characterized in that said elastic pad (14) has a height comprised between 8 and 12 millimeters.

12-13. (canceled)

14. The wearable device (10) for treating carpal tunnel syndrome according to claim 1, characterized in that said main body (12) comprises a lower opening (26) which is adapted for the passage of said hand (30).

15. The wearable device (10) for treating carpal tunnel syndrome according to claim 1, characterized in that said main body (12) comprises an upper end (28), which comprises at least one upper opening which is adapted for the passage of the fingers of said hand (30).

16. The wearable device (10) for treating carpal tunnel syndrome according to claim 15, characterized in that said upper end (28) of said main body (12) comprises an upper opening (32) that is adapted for the passage of the thumb of said hand (30).

17. The wearable device (10) for treating carpal tunnel syndrome according to claim 10, characterized in that said upper end (28) of said main body (12) comprises five upper openings (32, 34, 36, 38, 40).

18. The wearable device (10) for treating carpal tunnel syndrome according to claim 1, characterized in that said main body (12) further comprises an extractor (37), in the form of a tab or a flap, adapted to facilitate the extraction of said wearable device (10) from said hand (30).

19. The wearable device (10) for treating carpal tunnel syndrome according to claim 1, characterized in that said back (54) of said main body (12) is made of spandex.

20-21. (canceled)
